(19) United States
(12) Patent Application Publication
(10) Pub. No.: US 2006/0041017 A1
(43) Pub. Date: Feb. 23, 2006

(54) Synergistic conjugated linoleic acid (CLA) and carnitine combination

(76) Inventor: Raj K. Chopra, Westbury, NY (US)

Correspondence Address: COLEMAN SUDOL SAPONE, P.C., 714 COLORADO AVENUE, BRIDGE PORT, CT, 06605-1601, US

(21) Appl. No.: 11/201,014

(22) Filed: Aug. 10, 2005

Related U.S. Application Data
(60) Provisional application No. 60/603,165, filed on Aug. 20, 2004.
(60) Provisional application No. 60/630,410, filed on Nov. 23, 2004.

(51) Int. Cl. A61K 31/22; A61K 31/202

(52) U.S. Cl. 514/546; 514/560

(57) ABSTRACT

The present invention relates to the unexpected discovery that a combination of effective amounts of carnitine (in any form, as described in further detail herein) and conjugated linoleic acid (CLA) administered to a patient in need thereof exhibits synergistic activity in treating obesity by reducing fat mass and overall weight as well as one or more of hyperlipidemia, hypercholesterolemia, diabetes (both diabetes mellitus I and II), metabolic syndrome (syndrome X), kidiney failure and high blood pressure, where those conditions exist.

### RELATED APPLICATIONS

This application claims the benefit of priority from U.S. provisional application Ser. No. 60/603,165, filed Aug. 20, 2004 and Ser. No. 60/630,410, filed Nov. 23, 2004, both of which applications are incorporated by reference in their entirety herein.

### FIELD OF THE INVENTION

The present invention relates to novel compositions and methods for the treatment of obesity and secondary affects associated with obesity using carnitine or a pharmaceutically acceptable salt or derivative thereof in combination with conjugated linoleic acid.

### BACKGROUND OF THE INVENTION

Both conjugated linoleic acid (“CLA”) and carnitine are naturally occurring compounds. Carnitine is widespread in nature and can be found in all organs of mammals and many lower forms of animals and also in many microorganisms and plants (Fraenkel and Friedmann, 1957). Its chemical structure was identified in the late 1920s as 3-hydroxy-4-N,N, N-trlmethylaminobutyrate, Carnitine occur s in two enantiomeric forms, the natural carnitine being the L-form (Robouche and Seim, 1998: Borum and Bennett, 1988). Since meat and dairy products are the major dietary sources of carnitine (Rebouche and Engel, 1984), strict vegetarians are at risk for carnitine deficiency.

CLA occurs naturally as a mixture of cis-trans isomers in dairy products, ground beef and other meat products (Belury, 1995). The chemical name for CLA is octadecadienoic acid. Of the different CLA isomers known, just two have been linked specifically to health benefits. One is called cis-9, trans-11 (c9-t11), and the other, trans-10, cis-12 (t10-c12). Since isolating each of these isomers is difficult and expensive, most studies have therefore been conducted with a mixture of the two that is commercially produced from vegetable oil.

Although linoleic acid, a precursor of CLA, is recognized as an essential fatty acid, CLA is not considered a dietary essential. At doses achievable in an ordinary diet, CLA does not appear to provide any of the reported health benefits, several research teams have undertaken attempts to boost the natural concentrations of c9-t11 in meat and dairy products, but even enriched foods may not contain enough CLA to deliver the desired effect. The beneficial effects of CLA become evident when it is administered at higher doses as in a dietary supplement.

Carnitine is synthesized in the body from two amino acids, namely L-lysine and L-methionine. Although some refer to carnitine as an amino acid, it is not in the true sense of the word, nor is it a structural component of any protein. Although carnitine occurs and is synthesized in our body, under certain physiologic conditions the synthesis falls short of meeting the needs, and therefore carnitine is considered a “conditionally essential” and a “vitamin-like” nutrient (Borum and Bennett, 1986). In this regard, it is similar to nutrients such as coenzyme Q₁₀, choline and taurine.

Carnitine has many important metabolic functions (Borum and Bennett, 1986, Robouche and Seim, 1998). It plays a critical role in fat metabolism. It is essential for transporting long-chain fatty acid molecules (acylCoA) from the cytosol into the mitochondria of cells in the form of acylcarnitine. The acylcarnitine is then shuttled across the inner mitochondrial membrane where it is reconjugated to form acylCoA. The free carnitine molecule is then shuttled back to pick up another acyl moiety. This specialized transport mechanism which is tightly regulated is known as the “carnitine shuttle”, Thus, carnitine supplies the necessary fuel for oxidation in the electron transport chain to produce vital biological energy as ATP (adenosine triphosphate).

Although several beneficial effects of CLA have been reported, the exact mechanisms of actions of CLA are not clearly understood (Pariza et al, 2000). There are several reports on the favorable effect of CLA on body composition, with particular reference to reducing body fat and promote lean body mass (Ostrowski et al, 1999, Blankson et al, 2000, Gavino at al, 2000, Rahman et al, 2001). Reduction in leptin secretion appears to be one of the mechanisms for this effect (Kang and Pariza, 2001). Because of this property, there are numerous products for weight loss on the market that now contain CLA. Among the other potential uses for CLA are hypercholesterolemia, cardiovascular disease, triglyceridemia, type 2 diabetes and several types of cancers (Belury, 1995, Sebedlo et al, 2000, P D R for Nutritional supplements, 2001).

The rationale for a product containing a combination of carnitine and CLA lies in the fact that there is a close metabolic interrelationship between the two structurally dissimilar molecules in several pathways. Many of their biological effects are either similar or complementary. One such example is their respective roles in fat metabolism, as indicated earlier, carnitine transports the fatty acids for beta oxidation in the mitochondria eventually producing vital biological energy in the form of ATP. CLA has been shown to facilitate this process of fatty acid transport by enhancing the activity of the enzyme carnitine-palmitoyltransferase (Park at al. 1997, Rahman at al, 2001). Furthermore, data show that CLA also stimulates the beta oxidation of fatty acids (de Deckere et al, 1999). The beneficial effects of CLA on body composition appear to be due in part to its effect on lipid metabolism by way of reducing fat deposition and increasing lipolysis in adipocytes, possibly coupled with enhanced fatty acid oxidation in both muscle cells and adipocytes (Park et al, 1997), There is evidence to show that both carnitine and CLA improve cardiovascular function and reduce the risk for atherosclerosis (Lee et el, 1994, Retter, 1999) and these effects could be partly attributed to their favorable effects on fat metabolism.

Both carnitine and CLA have also been found to be effective in enhancing carbohydrate metabolism. They have been shown to improve impaired glucose tolerance. They augment the aerobic metabolism of carbohydrates and also enhance oxidative phosphorylation (Schonekess et al, 1995, Houseknecht et al, 1998). These findings have a direct bearing in the treatment of diabetes (McCarty, 2000). In addition, both carnitine and CLA possess antioxidant activities, thus indicating that carnitine and CLA may be useful where general antioxidant properties are indicated.

### OBJECTS OF THE INVENTION

It is an object of the invention to provide synergistic combinations of carnitine and CLA which may be used to treat the symptoms of obesity, and with it, favorable impact on diabetes mellitus type I and II, high glucose levels, hyperlipidemia, hypercholesterolemia, metabolic syndrome X, kidney failure and high blood pressure.

It is a further object of the invention to provide methods of treating a patient or subject for any one or more of obesity, diabetes mellitus type I and II, high glucose levels, hyperlipidemia, hypercholesterolemia, metabolic syndrome X, kidney failure and high blood pressure.

One or more of these and/or other objects of the invention may be readily gleaned from a review of the description of the invention which follows.

### BRIEF DESCRIPTION OF THE INVENTION

The present invention relates to the unexpected discovery that a combination of effective amounts of carnitine (in any form, as described in further detail herein) and conjugated linoleic acid (CLA) administered to a patient in need thereof exhibits synergistic activity in treating obesity by reducing fat mass and overall weight as well as one or more of hyperlipidemia, hypercholesterolemia, diabetes (both diabetes mellitus I and II), metabolic syndrome X, kidney failure and high blood pressure.

The methods comprise administering to a patient or subject in need thereof an effective amount of carnitine and CLA in combination for a period sufficient to produce a substantial alleviation in the symptoms associated with one or more of the conditions of obesity, including, hyperlipidemia, hypercholesterolemia, diabetes, metabolic syndrome X, kidney failure or high blood pressure.

### BRIEF DESCRIPTION OF THE FIGURES

FIG. 1 shows the body weight of rats which occurred after the two week administration of CLA, carnitine, CLA+carnitine or for control rats as described in Examples 4 and 5.

FIG. 2 shows the CPT activity as a function of control, CLA, carnitine or CLA+carnitine. These figures with results provide evidence of the synergistic effect of CLA and carnitine on weight loss as well as CPT activity, as described in examples 4 and 5.

### DETAILED DESCRIPTION OF THE INVENTION

The following terms shall be used to describe the present invention.

The term “patient” or “subject” is used throughout the specification to describe an animal, generally a mammalian animal, including a human, to whom treatment or use with the compounds or compositions according to the present invention is provided. For treatment or use with/or of those conditions or disease states which are specific for a specific animal (especially, for example, a human subject or patient), the term patient or subject refers to that particular animal.

The term “effective amount” refers to the amount of a selected carnitine compound and/or CLA which produces an intended result within the context of its/their administration, which relates to providing a synergistic favorable impact on obesity, manifest in the loss of at least 1% of the body weight of the subject or a reduction in the body mass of a subject of at least about 0.5%, at least about 1% at least about 2%, at least about 3%, at least about 4% and at least about 10% within a period of no greater than about 6 months, preferably no greater than about 4 months, more preferably no greater than about 2 months without a significant change in the lifestyle of the subject (i.e., a lifestyle change which, when compared to an original lifestyle, materially impacts weight loss). This definition should be considered exemplary and not limiting, however, as the body weight of a given subject pursuant to the present method may see an increase or decrease in body weight, in the event that significant lifestyle changes accompany the present methods. For example, if a subject were to initiate a rigorous weight-lifting or heavy exercise program to build muscle, that lifestyle change itself may result in a greater overall body weight, even as fat as a percentage of body weight is reduced significantly (lower body mass index). The precise amount of each of the carnitine compound and/or CLA used in the present invention will vary depending upon the particular compound selected and its intended use, the age and weight of the subject, route of administration, and so forth, but may be easily determined by routine experimentation. In the case of the treatment of a condition or disease state, an effective amount is that amount which is used to effectively treat the particular condition or disease state, i.e., to substantially reduce the symptoms associated with that condition or disease state. In the present invention, preferred effective amounts are those which produce a synergistic effect in treating the particular disease state or condition.

The term “pharmaceutically acceptable carrier” refers to an additive, carrier or excipient which is not unacceptably toxic to the subject to which it is administered. Pharmaceutically acceptable excipients are described at length by E. W. Martin, in “Remington's Pharmaceutical Sciences.”

The term “synergistic” is used to describe the activity of a combination of carnitine and CLA according to the present invention in that the effect is more than the added activity expected when these two compounds are administered in combination to treat a particular condition or disease state. In its primary feature, the present invention relates to the treatment of obesity, therefore weight loss or fat reduction is a key feature.

The term “without a significant change in lifestyle” is used to describe the lifestyle of a patient or subject before administration of a CLA/carnitine combination according to the present invention in comparison to that patient's lifestyle after commencing administration of a CLA/carnitine combination according to the present invention. This term is used to distinguish effects on obesity in a patient or subject where a change in that patient's or subject's lifestyle materially contributes to an impact on obesity.

The terms “coadministered” and “administered in combination” are used to describe the administration of carnitine and CLA in the present invention. In the present invention carnitine and CLA are administered in effective amounts. Such term signifies that the blood levels of carnitine and CLA may be found in the patient or subject which are consistent with the activity for which these compounds are administered. The terms including administration of effective amounts of carnitine and CLA are the same time or at different times on the same day in order that effective amounts of both of these compounds are administered to a patient or subject during a single day.

The term “obesity” refers to a condition of a subject or patient or subject who has excess adipose tissue or fat well above normal with a body-mass index of at least about 31 and in certain cases greater than 40. Slightly obese subjects or patients have a body-mass index of ranging from about 31-35, those who are moderately obese have a body mass index ranging from about 36-40 and those who are severely obese have a body-mass index greater than 40.

Obese patients or subjects have an increased risk for type II diabetes, hypertension and cardiovascular disease. Obese individuals often suffer from one or more of the following complications: metabolic complications including high cholesterol, syndrome X, platelet dysfunction, thromboembolic disease, fatty liver disease (nonalcoholic steatohepatitis), gallstones, pancreatitis, reproductive dysfunction (irregular menstrual cycles and infertility), central hypoventilation syndrome, asthma, nutritional deficiencies; structure complications, including obstructive sleep apnea, gastroesophageal reflux disease, asthma associated with gastroesophageal reflux disease, venous insufficiency, venous thrombosis, pseudotumor cerebri, skin infections and ulcers, poor wound healing, stress incontinence, injuries, surgical complications; degenerative complications, including axial arthritis, osteoarthritis, vertebral disk disease, atherosclerotic cardiovascular disease, stroke, complications of diabetes, kidney disease, kidney failure, left-ventricular hypertrophy, right-sided heart failure, cirrhosis associated with nonalcoholic steatohepatitis, Alzheimer's disease; neoplastic conditions/diseases including cancers such as endometrial, breast, ovarian, cervical, prostate, colorectal, esophageal adenocarcinoma (secondary to gastroesphageal reflux), gallbladder, pancreatic, renal cell; psychological conditions/diseases including depression, anxiety and panic, negative self-image, feelings of inadequacy, binge eating and reactive bulimia, among others, including complications during pregnancy including increased risk of death in mother and baby, increased risk of maternal high blood pressure, increased risk of mother developing gestational diabetes and increased risk of complications during labor and delivery. In addition, infants born to women who are obese during pregnancy are more likely to be high birthweight, may face a higher rate of C-section delivery and low blood sugar (which can be associated with brain damage and seizures), as well as an increased risk of birth defects, in particular, neural tube defects like spina bifida.

The term “diabetes” is used to describe diabetes mellitus, types I and II. Type I diabetes mellitus, is an insulin-dependent diabetes mellitus, and type II diabetes mellitus, is a non-insulin-dependent diabetes mellitus. Type I diabetes mellitus is a metabolic disease in which carbohydrate utilization is reduced and which is caused by an absolute or relative deficiency of *insulin. It is characterized by hyperglycemia, glycosuria, water and electrolyte loss, ketoacidosis and coma. Type II diabetes mellitus generally occurs in people who are over 35, where glucose tolerance is low and where plasma insulin and glucose levels are elevated. It occurs quite often in people who are obese. The present invention is useful for the treatment of both type I and type II diabetes mellitus, more preferably type II diabetes mellitus.

The term “high blood pressure” is used to describe a condition or disease state where the blood pressure of a patient or subject is at least 15-20% above the normal blood pressure range for diastolic and/or systolic blood pressure readings.

The term “metabolic syndrome X” refers to disturbances in metabolic pathways in a pateitn such that metabolism of free fatty acids and sugars is substantially decreased and fat tissue exhibits an increase in weight.

The term “carnitine” or “derivative of carnitine” is used to describe a compound according to the general structure: 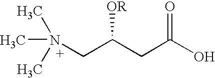
where R is H or a C₂ to C₂₀ straight or branch-chained alkanoyl group, preferably a C₂ to C₈ straight or branch-chained alkanoyl group, even more preferably a C₂ to C₅ straight or branch-chained alkanoyl group, or a pharmaceutically acceptable salt thereof. Preferred carnitine compounds according to the present invention include carnitine, acetyl-, an ester of L-carnitine, such as propionyl-, butyryl, valeryl and isovaleryl L-carnitine. Acceptable carnitine derivatives for use in the present invention include, for example, those carnitine derivatives which are described in U.S. Pat. Nos. 6,703,042; 6,610,699; 6,476,243; 6,465,515; 6,124,360; 6,051,608 and 5,998,474, relevant portions of which are incorporated by reference herein. Note that in context, the term carnitine may refer to the specific compound (R is H) or to one or more of carnitine or its derivative or pharmaceutically acceptable salt.

The term “conjugated linoleic acid” or “CLA” refers to any conjugated linoleic acid or octadecadienoic acid and its salts and derivatives. It is intended that this term encompass and indicate all positional and geometric isomers of linoleic acid with two conjugated carbon-carbon double bonds any place in the molecule. CLA differs from ordinary linoleic acid in that ordinary linoleic acid has double bonds at carbon atoms 9 and 12. Examples of CLA include cis- and trans isomers (“E/Z isomers”) of the following positional isomers: 2,4-octadecadienoic acid, 4,6-octadecadienoic acid, 6,8-octadecadienoic acid, 7,9-octadecadienoic acid, 8,10-octadecadienoic acid, 9,11-octadecadienoic acid and 10,12 octadecadienoic acid, 11, 13 octadecadienoic acid. As used herein, CLA encompasses a single isomer, a selected mixture of two or more isomers, and a non-selected mixture of isomers obtained from natural sources, as well as synthetic and semisynthetic CLA. As used herein, CLA further encompasses free fatty acid(s) of CLA, physiologically acceptable salts of CLA, and esters with physiologically acceptable, preferably naturally occurring, alcohols (e.g., ethanol and glycerol), and CLA triglycerides.

As used herein, it is intended that “triglycerides” of CLA contain an isomer of CLA at any or all of three positions on the triglyceride backbone. Methods for the synthesis of triglycerides containing CLA are taught in PCT Application US99/05806, which is incorporated by reference herein.

As used herein, the term conjugated linoleic acid or CLA is intended to include “esters” of CLA which term includes any CLA isomer bound through an ester linkage to an alcohol or any other chemical group. Methods for the synthesis of esters containing CLA are taught in PCT Application US99/05806, incorporated herein by reference.

CLA includes both major and minor isomers, including, for example, c11,t13; t11,c13; t11,t13; and c11,c13 octadecadienoic acid.

As used herein, “c” encompasses a chemical bond in the cis orientation, and “t” refers to a chemical bond in the trans orientation. If a positional isomer of CLA is designated without a “c” or a “t”, then that designation includes all four possible isomers. For example, 10,12 octadecadienoic acid encompasses c10,t12; t10,c12; t10,t12; and c10,c12 octadecadienoic acid.

In certain embodiments of the invention, the conjugated linoleic acid administered to patients in combination with L-carnitine or salt or derivative thereof is an octadecadienoic acid isomer selected from the group of cis-9, trans-11; cis-9, cis-11; trans-9, cis-11; trans-9, trans-11; cis-10, cis-10, trans-12; trans-10, cis-12; trans-10, trans-12 octadecadienoic acid and mixtures thereof. In other embodiments, the conjugated linoleic acid administered to patients contains less than 5% of minor isomers of conjugated linoleic acid. In still other embodiments, the minor isomer is c11,t13; t11,c13; t11,t13; or c11,c13 octadecadienoic acid. In a particularly preferred embodiment, the conjugated linoleic acid contains less than 1% of minor isomers of conjugated linoleic acid. In still other embodiments, the conjugated linoleic acid further comprises an ester or triglyceride. In still other embodiments, the conjugated linoleic acid further comprises greater than about 55% t10,c12 octadecadienoic acid.

In preferred embodiments, preferred embodiments, the CLA of the present invention comprises a mixture of one or all of the isomers of octadecadienoic acid including the cis-9, trans-11; cis-9, cis-11; trans-9, cis-11; trans-9, trans-11; cis-10, cis-12; cis-10, trans-12; trans-10, cis-12; and trans-10, trans-12 isomers. The rearrangement of the double bonds of linoleic acid to conjugated positions has been shown to occur during treatment with catalysts such as nickel or alkali at high temperatures, and during autooxidation. Theoretically, eight possible geometric isomers of 9,11 and 10,12 octadecadienoic acid (i.e., c9,c11; c9,t11; t9,c11; t9,t11; c10,c12; c10,t12; t10,c12 and t10,t12) would form from the isomerization of c9,c12-octadecadienoic acid.

A general mechanism for the isomerization of linoleic acid was described by Cowan (Cowen, JAOCS, 72:492-99 (1950)). Although an understanding of the mechanism is not required for the practice of the present invention, it is believed that the double bond is polarized by the result of a collision with an activating catalyst. The polarized carbon atom and its adjoining carbon are then free to rotate and the forces are such as to make the deficient carbon atom essentially planar. When the system reacts to relieve these forces set up as a result of the collision, both cis and trans isomers are formed. The formation of certain isomers of CLA is thermodynamically favored. This is due to the co-planar characteristics of the five carbon atoms around the conjugated double bond and a spatial conflict of the resonance radical.

Although an understanding of this mechanism is not required for the practice of the present invention, the relatively higher distribution of 9,11 and 10,12 isomers apparently results from the further stabilization of the c9,t11 or t10,c12 geometric isomers. The cis-9,trans-11 and trans-10, cis-12 isomers are thought to have the most biological activity. Therefore, in preferred embodiments, these isomers may be used in a purified form, or in CLA compositions containing high ratios of these isomers. In addition, methods for manufacturing for example, CLA 80 are provided in the literature, for example, Example 2 of U.S. Pat. No. 6,440,931 (i.e., low temperature nonaqueous alkali isomerization) and an alternative method of manufacturing another preferred CLA composition is provided in Example 3 of that patent (i.e., isomerization with alkali alcoholate in the presence of a monohydric low molecular weight alcohol). Both methods provide for the production of CLA predominantly comprising the c9,t11- and t10,c12-isomers, with low levels of 8,10-,11,13- and trans-trans isomers. In preferred embodiments of the present invention, CLA mixtures contain less than about 5% of minor CLA isomers; while in particularly preferred embodiments, the present invention utilizes CLA with less than about 1% of minor CLA isomers. Preferred isomers in the CLA mixtures include 9,11-octadecadienonic acid, 10,12-octadecadienoic acid, most preferably the c9,t11 and t10,c12 isomers. In other preferred embodiments, the mixture contains greater than about 50% t10,c12 isomer. In other particularly preferred embodiments, the mixture contains greater than about 55% t10,c12 isomer. In a particularly preferred embodiment, the mixture contains greater than about 60% t10,c12 isomer. It is contemplated that in some embodiments, supplementation of the mixture derived from isomerization of linoleic acid with purified or synthesized t10,c12 isomer may be necessary to achieve these percentages.

The present invention also contemplates the use of derivatives of CLA. The term “CLA” shall also includes such derivatives in context. For example, CLA may be free or bound through ester linkages or provided in the form of an oil containing CLA triglycerides. In these embodiments, the triglycerides may be partially or wholly comprised of CLA attached to a glycerol backbone. The CLA may also preferably be provided as a methylester or ethylester. Furthermore, the CLA may be in the form of a non-toxic salt, such as a potassium or sodium salt (e.g., a salt formed by reacting chemically equivalent amounts of the free acids with an alkali hydroxide at a pH of about 8 to 9).

In a preferred embodiment of the present invention, a safe and effective amount of a combination of carnitine and CLA is orally administered to an obese patient or subject in order to assist in reducing the patient's body fat mass (body mass index or BMI) and overall weight, hyperlipidemia, hypercholesterolemia, glucose levels, high blood pressure and the symptoms of diabetes (both diabetes mellitus I and II) and metabolic syndrome (syndrome X) by providing more efficient metabolism of fat and glucose. The use of carnitine and CLA for the treatment of obesity indications is desirable because both carnitine and CLA are non-toxic, naturally occurring food ingredients. Carnitine and CLA are not classified as a drug and may be consumed as a part of a normal diet and finds use as a part of everyday nutrition.

Effective amounts of carnitine and CLA are used in combination in the present invention. The amount of carnitine administered per day to a subject will generally range from about in combination 50 mg to about 5 grams or more, more preferably about 100 mg to about 3 grams, and even more preferably at least about 500-1000 mg within this range. An effective amount of CLA to be used in combination with carnitine will range from about 250 mg to about 25 grams per day, more preferably about 500 mg to about 5-10 grams per day. This combination of carnitine and CLA will provide a synergistic effect to substantially reduce obesity or one or more of the deleterious conditions associated with obesity as well as diabetes mellitus type I or II, hyperlipidemia, high blood glucose levels, hypercholesterolemia, syndrome X and/or high blood pressure. The ratio of CLA to carnitine (one a weight by weight basis) in methods and compositions according to the present invention preferably ranges from about 10:1 to about 1:3, with a preferred ratio of about 6:1 to about 1:2, of about 5:1 to about 1:1, about 4:1 to about 2:1 and about 3:1. In particularly preferred embodiments of the present invention, the administration of carnitine in combination with CLA results in no detrimental effects in patients.

The administration of CLA and carnitine may be at the same time or at different times provided that effective concentrations of both CLA and carnitine are both found in the subject at at the same time. Preferably, administration of both CLA and carnitine is at the same time, preferably in a single composition, in order to facilitate the compliance of the subject to adhere to a schedule of administration.

It is contemplated that there will be some variation in effectiveness due to differences among individuals in physiological and biochemical parameters (e.g., body weight and basal metabolism), exercise, and other aspects (e.g., diet). In preferred aspects, it is contemplated that individuals beginning treatment will be given a 1.0 gram dose of carnitine in combination with a 3.0 gram dose of CLA daily for an initial two month period, and then, if no reduction of obesity or the secondary conditions associated with obesity are improved, gradually increase the carnitine dose above 1 gram per day and the CLA dose up to about 6 grams per day or higher.

In a preferred embodiment, administration is oral. The CLA and carnitine may be formulated together with suitable carriers such as starch, sucrose or lactose in tablets, pills, dragees, capsules, solutions, liquids, slurries, suspensions and emulsions. The carnitine and CLA may be administered separately or together, provided that the total amount of CLA and carnitine is an effective amount in combination per day to have a substantial impact on obesity. In preferred aspects, the use of the present invention results in at least about a 1-2% body weight reduction, at least about a 2-3% body weight reduction, preferably at least about a 5% body weight reduction and in certain instances at least about 10% body weight reduction of the subject after a period of no greater than about two months. These weight reductions translate generally into a reduction of the body mass index (BMI) of at least 0.5%, preferably at least about 1% up to about 5-10% or more.

Additionally, carnitine and CLA may be provided in aqueous solution, oily solution, as a powder, or in any of the other forms discussed above. The tablet or capsule of the present invention may be coated with an enteric coating which dissolves at a pH of about 6.0 to 7.0. A suitable enteric coating which dissolves in the small intestine but not in the stomach is cellulose acetate phthalate. Sustained or controlled release versions of the present formulations are contemplated. In a preferred formulation, the carnitine and CLA is provided in soft gelatin capsules containing about 250 mg of carnitine and about 500-750 mg CLA. Alternatively, a formulation comprising about 500 mg carnitine and about 1000-1500 mg of CLA may be preferred. Other formulations may contain, for example, about 100 mg of carnitine and about 200-250 mg CLA, 50 mg of carnitine and 100-150 mg of CLA, or 750 mg of carnitine and 1500-2500 mgCLA. In another preferred embodiment, the carnitine and CLA together are provided as a powder contained in a capsule. The carnitine and CLA may also be provided by any of a number of other routes, including, but not limited to, intravenous, intramuscular, intra-arterial, intramedullary, intrathecal, intraventricular, transdermal, subcutaneous, intraperitoneal, intranasal, enteral, topical, sublingual or rectal means and dosage forms consistent with this delivery are contemplated by the present invention. Further details on techniques for formulation for and administration and administration may be found in the latest edition of Remington's Pharmaceutical Sciences (Maack Publishing Co., Easton, Pa.).

An effective amount of carnitine and CLA may also be provided as a supplement in various prepared food products and drinks. For the purposes of this application, prepared food product means any natural, processed, diet or non-diet food product to which carnitine and CLA have been added. The carnitine may be added in any form, including free form, as an ester (alkanoyl form) or as pharmaceutically acceptable salts. CLA may be added in the form of free fatty acids or as an oil containing partial or whole triglycerides of CLA. Carnitine and CLA may be directly incorporated into various prepared food products, including, but not limited to diet drinks, diet bars, supplements, prepared frozen meals, candy, snack products (e.g., chips), prepared meat products, milk, cheese, yogurt and any other fat or oil containing foods. Preferably, carnitine and CLA are provided together in oral dosage form, even more preferably, in capsule form such as a hard capsule or preferably a soft gelatin capsule.

In some preferred embodiments, CLA and carnitine is used in combination with anti-hyperglycemic agents. Examples of such agents with which CLA can be combined include insulin, metformin, chorplopamide, glipizid, glibenclamide and/or acarbose. In still other embodiments, carnitine and CLA may be used in combination with vanadium compounds, chromium compounds, lipoic acid, AGE inhibitors/breakers or other compounds with known positive effect on relieving the symptoms associated with the conditions or disease states associated with obesity, metabolic syndrome (syndrome X) and/or high blood pressure.

Carnitine and CLA are somewhat susceptible to oxidation. Therefore, it is desirable to package CLA and carnitine or its derivative for human use with suitable antioxidants such as lecithin, tocopherols, ascorbate, ascorbyl palmitate or spice extracts such as rosemary extract.

The following examples are provided to support the present invention. They should not be viewed as limiting the present invention in any way.

### EXAMPLES

### Example 1

### CLA-Carnitine Softgel Composition

| | Amount Per | | Daily Dose | |
|---|---|---|---|---|
| Ingredient: | Softgel | | (6 Softgels) | |
| CLA (Clarinol A-95)* | 167 | mg. | 1000 | mg |
| L-Carnitine Base** | 84 | mg | 500 | mg |
| High P.C. Lecithin | 50 | mg | 300 | mg |
| Beeswax | 12.65 | mg | 76 | mg. |
| MCT | 386.35 | | 2318 | mg |
| (Medium Chain Triglycerides) | | | | |
| Total: | 700 | mg | | |

Method: Make a blend of the above ingredients to homogenize same and encapsulate using standard softgel methodology.
Recommended Daily Dose: Two softgels, three times daily with meals.
*Clarinol 95 may be substituted with any other pharmaceutically acceptable product containing conjugated linoleic acid (for example, TONALIN).
**Note that L-carnitine base may be substituted with available salts of L-carnitine, such as tartarate, fumarate, citrate, orotate, etc. It can also be replaced by L-carnitine esters, such as Acetyl-L-carnitine, Propionyl L-carnitine, and their salts and derivatives thereof, as otherwise described herein.

### Example 2

### CLA-Carnitine Softgel Composition

| | Amount Per | | Daily Dose | |
|---|---|---|---|---|
| Ingredient: | Softgel | | (6 Softgels) | |
| CLA (Clarinol A-95)* | 584 | mg. | 3500 | mg |
| L-Carnitine Base** | 167 | mg | 1000 | mg |
| High P.C. Lecithin | 50 | mg | 300 | mg |
| Beeswax | 12.65 | mg | 76 | mg. |
| MCT | 286.35 | | 1718 | mg |
| (Medium Chain Triglycerides) | | | | |
| Total: | 1100 | mg | | |

Method: Make a blend of the above ingredients to homogenize same and encapsulate using standard softgel methodology.
Recommended Daily Dose: Two softgels, three times daily with meals.
*Clarinol 95 may be substituted with any other pharmaceutically acceptable product containing conjugated linoleic acid (for example, TONALIN).
**Note that L-carnitine base may be substituted with available salts of L-carnitine, such as tartarate, fumarate, citrate, orotate, etc. It can also be replaced by L-carnitine esters, such as Acetyl-L-carnitine, Propionyl L-carnitine, and their salts and derivatives thereof, as otherwise described herein.

### Example 3

### CLA-Carnitine Softgel Composition

| | Amount Per | | Daily Dose | |
|---|---|---|---|---|
| Ingredient: | Softgel | | (6 Softgels) | |
| CLA (Clarinol A-95)* | 834 | mg. | 5000 | mg |
| L-Carnitine Base* | 250 | mg | 1500 | mg |
| High P.C. Lecithin | 50 | mg | 300 | mg |
| Beeswax | 12.65 | mg | 76 | mg. |
| MCT | 153.35 | | 920 | mg |
| (Medium Chain Triglycerides) | | | | |
| Total: | 1300 | mg | | |

Method: Make a blend of the above ingredients to homogenize same and encapsulate using standard softgel methodology.
Recommended Daily Dose: Two softgels, three times daily with meals.
*Clarinol 95 may be substituted with any other pharmaceutically acceptable product containing conjugated linoleic acid (for example, TONALIN).
**Note that L-carnitine base may be substituted with available salts of L-carnitine, such as tartarate, fumarate, citrate, orotate, etc. It can also be replaced by L-carnitine esters, such as Acetyl-L-carnitine, Propionyl L-carnitine, and their salts and derivatives thereof, as otherwise described herein.

### Examples 4 and 5

Two experiments were carried out to determine the potential synergistic effect of conjugated linoleic acid (CLA)—carnitine combination using adult rats. The objective was to examine their effect on the activity of the enzyme carnitine palmitoyl transferase (CPT 1). in brown adipose tissue. Brown adipose tissue is an important site for long-term storage of fat in the body and for the rapid metabolism of fat for thermogenesis. CPT I is a key enzyme in this process and it has an essential role In the metabolism of fats by transporting long chain fatty acids into the Inner mitochondria for oxidation.

The first experiment involved the following groups of adult rats. The treatment involved administration of the following products daily by oral gavage for two weeks, the control group receiving water. Carnitine was administered as the fumarate salt.
1. Control (water)−(1 mL)
2. L-Carnitine−273 mg (in 1 mL water)
3. CLA−354 mg (In 1 mL corn oil)
4. Carnitine+CLA−273 mg+364 mg (in 1 mL corn oil)

At the end of two weeks, the animals were euthanized and brown adipose tissue was excised for CPT I assay according to Rahman et al(2001).

A second experiment was carried out using much lower doses of products (20% of the original dose) using the same experimental design, except that the control group received corn oil instead of water.
1. Corn Oil−(1 mL)
2. L-Carnitine−68.25 mg (in 1 mL water)
3. CLA−91 mg (in 1 mL corn oil)
4. Carnitine+CLA−68.25 mg+91 mg (in 1 mL corn oil)

The results are presented in Tables 1 and 2, below. The data clearly show a highly significant enhancement of CPT I activity by the carnitine-CLA combination, over and above the increase observed with carnitine or CLA alone. This synergistic effect of carnitine-CLA combination in facilitating the oxidation of fats is highly significant and is of immense clinical importance.

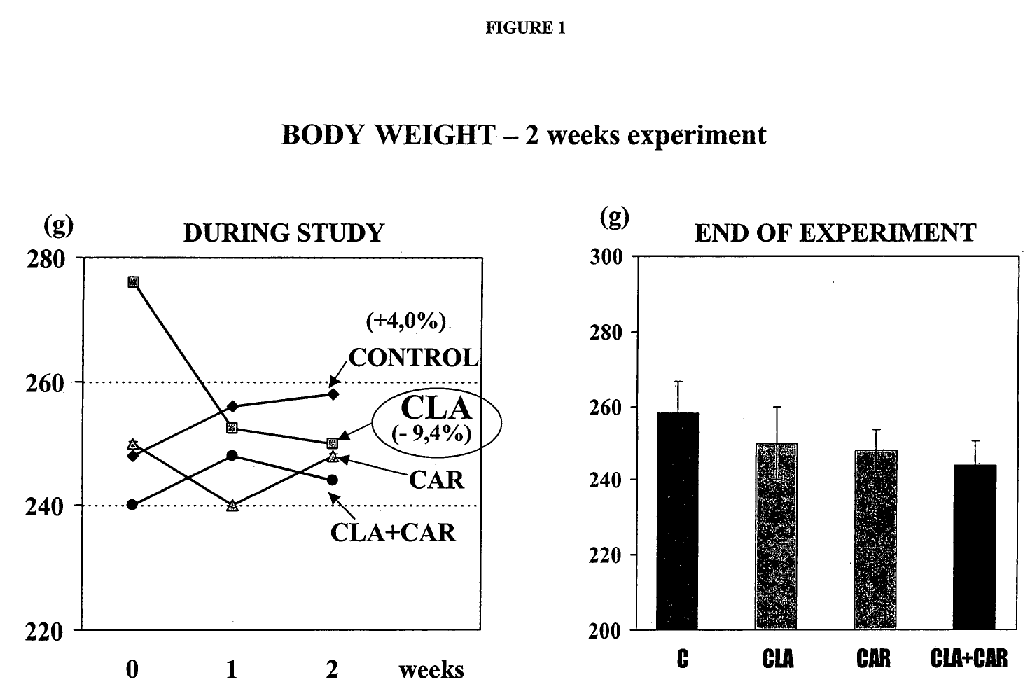

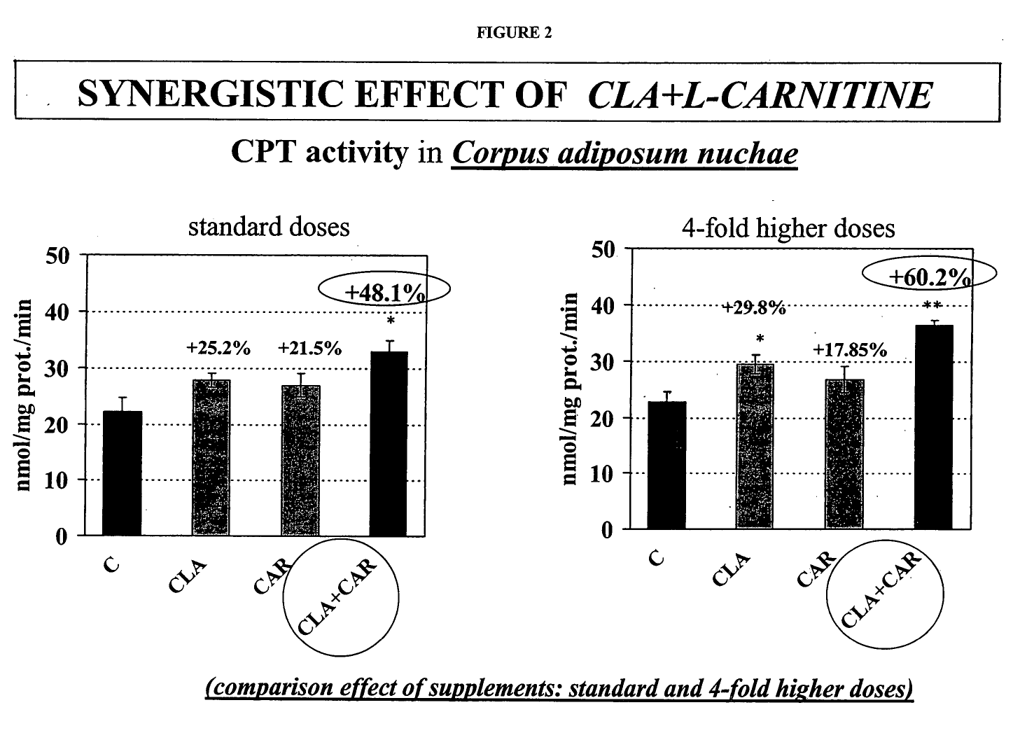

FIG. 1 shows the body weight of rats which occurred after the two week administration of CLA, carnitine, CLA+carnitine or for control rats. FIG. 2 shows the CPT activity as a function of control, CLA, carnitine or CLA+carnitine. These figures with results provide evidence of the synergistic effect of CLA and carnitine on weight loss as well as CPT activity.

TABLE 1
CPT I activity in brown adipose tissue (standard dose)
(nmoles/mg protein/min)(Mean ± SEM)

| | Control | | | CLA + |
|---|---|---|---|---|
| | (water) | Carnitine | CLA | Carnitine |
| N | 7 | 7 | 4 | 3 |
| Mean + SEM | 22.746 + 1.968 | 26.808 ± 2.373 | 29.530 + 1.590 | 36.440 ± 0.812 |
| P (vs. control) | — | NS* | 0.0443 | 0.0025 |
| % Control | 100 | 117.85 | 129.82 | 160.20 |

*Not significant

TABLE 2
CPT I activity in brown adipose tissue (low dose)
(nmoles/mg protein/min)(Mean ± SEM)

| | Control | | | |
|---|---|---|---|---|
| | (corn oil) | Carnitine | CLA | CLA + Carnitine |
| N | 4 | 5 | 4 | 4 |
| Mean + SEM | 22.237 ± 2.666 | 27.024 ± 2.074 | 27.840 ± 1.259 | 32.925 ± .020 |
| P (vs. control) | — | NS* | NS* | 0.0187 |
| % Control | 100 | 12520 | 121.53 | 148.11 |

*Not significant

### Further Examples

Obesity is a risk factor for cardiovascular diseases, atherosclerosis, type 2 diabetes and certain cancers. According WHO report, obesity occurs in 250 million people in the world. Conjugated linoleic acid (CLA), a co-6-polyunsaturated fatty acid, has been shown to affect body fat reduction and promoting lean body mass. L-carnitine (CAR) is an essential cofactor of fatty acids transport through inner mitochondrial membrane. Carnitine palmitoytransferase (CPT) activity can be used as a marker of body fat reduction. CPT catalyzes the transformation of long-chain fatty acid-CoA esters to acylcarnitine for transport across the mitochondrial membrane. Effect of CLA and CAR on mitochondrial function and coenzyme Q (CoQ) production can participate in their synergistic antiobesity mechanisms.

The aim of the study was to examine:
a/whether there is a synergistic relationship between CLA and CAR in reduction of body fat
b/whether CLA and CAR can modulate heart, kidney and liver mitochondrial CoQ content and oxidative phosphorylation function
c/to contribute to pathobiochemical mechanism of relationship between CoQ content and CPT activity in liver mitochondria.

Methods: Four groups of 3-months old Wistar rats were given the following treatments by oral gavage daily for 2 weeks: (1) C (control)−1 ml corn oil, (2) CLA−91 mg CLA (in corn oil), (3) CAR−68,25 mg L-carnitine fumarate (in corn oil), (4) CLA+CAR−91 mg CLA with 68,25 mg L-carnitine fumarate (in corn oil). Body weight and body fat weight (corpus adiposum nuchae, corpus adiposum, perirenal tissue, omentum majus) were measured. CoQ ₁₀ and CoQ₉ in isolated mitochondria were estimated by HPLC method. For evaluation of parameters of oxidative phosphorylation were used Gilson oxygraph records of respiratory chain by using Clark oxygen electrode (S₃— ADP stimulated respiration, S₄— basal respiration, OPR—rate of ATP production). A spectrophotometric assay was used for CPT activity.

Results: Significant synergistic effect of CLA and CAR was observed with respect to increased CPT activity in corpus adiposum nuchae. Heart and kidney mitochondrial CoQ content (CoQ_{10-OX}+CoQ_{9-OX}) was significantly stimulated with CLA, CAR and CLA+CAR in comparison with control group.

TABLE 1
Dietary supplements effect on heart mitochondrial CoQ levels and CPT activity

| HEART | CoQ_{10-OX} | CoQ_{9-OX} | CoQ₁₀ + CoQ₉ | CPT |
|---|---|---|---|---|
| mitochondria | (nmol/mg prot.) | (nmol/mg prot.) | (nmol/mg prot.) | (nmol/mg rot./min) |
| CONTROL | 0.277 ± 0.033 | 2.958 ± 0.251 | 2.968 ± 0.052 | 13.80 ± 1.198 |
| CLA | 0.343 ± 0.041 | 3.379 ± 0.261 | 3.722 ± 0.298* | 15.52 ± 1.080 |
| CAR | 0.452 ± 0.021*** | 4.692 ± 0.168*** | 5.147 ± 0.181*** | 12.36 ± 1.759 |
| CLA + CAR | 0.315 ± 0.011 | 3.808 ± 0.083* | 4.128 ± 0.09*** | 13.36 ± 1.530 |

Values are means ± s.e.m.
Statistical significance versus control:
*significant,
**very significant,
***extremly significant

Significantly increased heart mitochondrial CoQ levels in supplemented groups only slightly stimulated respiratory chain function and ATP production.

TABLE 2
Dietary supplements effect on kidney mitochondrial CoQ levels and CPT activity

| KIDNEY | CoQ_{10-OX} | CoQ_{9-OX} | CoQ₁₀ + CoQ₉ | CPT |
|---|---|---|---|---|
| mitochondria | (nmol/mg prot.) | (nmol/mg prot.) | (nmol/mg prot.) | (nmol/mg rot./min) |
| CONTROL | 0.098 ± 0.004 | 0.945 ± 0.039 | 1.043 ± 0.039 | 4.024 ± 0.830 |
| CLA | 0.122 ± 0.005** | 1.162 ± 0.102 | 1.284 ± 0.105* | 6.517 ± 0.351* |
| CAR | 0.138 ± 0.012* | 1.290 ± 0.119* | 1.471 ± 0.128* | 2.974 ± 0.466 |
| CLA + CAR | 0.126 ± 0.011 | 1.237 ± 0.164 | 1.456 ± 0.169* | 4.185 ± 0.241 |

Significantly increased kidney mitochondrial CoQ levels, NAD-linked respiratory chain function and ATP production (S₃, OPR) in supplemented groups in comparison with control group. In this study were proved strongly correlations in liver mitochondria between: a/CPT activity and total CoQ: Control (r=0.612, CLA (r=0.996, p<0.05), CLA+CAR (r=0.763), b/CPT and OPR of FAD-linked respiratory chain: Control (p=0.654), CLA (r=1.000, p<0.001), CAR (r=0.978, p<0.04), CLA+CAR (r=0.767), c/CPT and S₃ of FAD-linked respiratory chain: Control (r=0.611), CLA (r=0.99, p<0.09), CAR (r=0.946, p<0.015), CLA+CAR (r=0.685).

Conclusion: This pilot study evidenced the synergistic effect of conjugated linoleic acid with L-carnitine in body fat reduction as assessed by CPT activity and significant increase of CoQ production in heart and kidney mitochondria. Liver mitochondrial CoQ content strongly regulates mitochondrial carnitine palmitoyltransferase activity and energy production. This suggests that CLA and CAR could reduce the risk of obesity and obesity-related diseases.

### Human In Vivo Study

A ten (10) subject study lasting 12 weeks was carried out. It was an open label trial. All 10 subjects (6 male, 4 Female) ranging in age from 22 to 45 maintained their regular lifestyle while consuming 2 softgels of the CLA/carnitine recipe with each meal, three times a day. This provided 3000 mg CLA and 1000 mg of carnitine per subject per day.

The results are as follows:

| | Baseline | After 12 weeks |
|---|---|---|
| 1. 22 year old (M) | 165 lbs | 156 lbs |
| 2. 31 year old (F) | 190 lbs | 181 lbs |
| 3. 24 year old (M) | 160 lbs | 148 lbs |
| 4. 42 year old (M) | 210 lbs | 200 lbs |
| 5. 45 year old (M) | 154 lbs | 145 lbs |
| 6. 29 year old (F) | 147 lbs | 145 lbs |
| 7. 38 year old (F) | 172 lbs | 165 lbs |
| 8. 40 year old (M) | 232 lbs | 220 lbs |
| 9. 23 year old (M) | 147 lbs | 142 lbs |
| 10. 41 year old (F) | 139 lbs | 132 lbs |

Results: Significant weight drop was evidenced by protocol CLA/carnitine co-administration in all subjects.

### References

Belury M A, Conjugated linoleate: a polyunsaturated fatty acid with unique chemopreventive properties. Nutr Rev 1995; 53:83-9.
Blankson H, Stakkestad J A, Fagertun H, Thom E, Wadstein J, Gudmundsen O. Conjugated Ilnoleic acid reduces body fat mass in overweight and obese humans. J Nutr 2000; 130:2943-8.
Borum P R, Bennett S G. Carnitine as an essential nutrient. J Am Coll Nutr 1986; 5:177-82.
de Deckere E A, van Arnelsvoort J M, McNeill G P, Jones P. Effects of conjugated linoleic acid (CLA) isomers on lipid levels and peroxisome proliferation in the hamster. Br J Nutr 1999; 82:309-17.
Fraenkel G, Friedmann S. Carnitine. Vitam Horm 1957; 15:73-118,
Gavino V C, Gavino G, Leblanc M J, Tuchweber B. An isomeric mixture of conjugated linoleic acids but not pure cis-9, trans-1-octadecadienoic acid affects body weight gain and plasma lipids in hamsters. J Nutr 2000; 130:27-9.
Houseknecht K L, Vanden Heuvel J P, Moya-Camarena S Y, Portocarrero C P, Peck L W, Nickel K P, Belury M A. Dietary conjugated linoleic acid normalizes impaired glucose tolerance in the Zucker diabetic fatty fa/fe rat. Biochim Biophys Res Commun 1998; 244:678-82.
Kang K, Pariza M W. Trans-10, cis-12-conjugated linoleic acid reduces leptin secretion from 3T3-L1 adipocytes. Biochem Biophys Res Commun 2001; 287:377-82.
Lee K N, Kritchevsky D, Pariza M W. Conjugated Iinoleic acid and atherosclerosis In rabbits. Atherosclero 1994; 108:19-25.
McCarty M F. Toward a wholly nutritional therapy for type 2 diabetes. Med Hypoth 2000; 54:483-7.
Ostrowski E, Muralitharan M, Cross R F. Dietary conjugated Ilinoleic acids increase lean tissue and decrease fat deposition in growing pigs. J Nutr 1999:120:2037-42.
Pariza M W, Park Y, Cook M E. Conjugated linoleic acid and the control of cancer and obesity. Toxicol Sci 1999; 52:107-10.
Pariza M W, Park Y, Cook M E. Mechanisms of action of conjugated linoleic acid: evidence and speculation. Proc Soc Exp Biol Mad 2000; 223:8-13.
Park Y, Albright K J. Liu W, Storkson J M, Cook M E. Pariza M W, Effect of conjugated linoleic acid on body composition in mice. Lipids. 1997; 32:853-8.
PDR for Nutritional Supplements, First Edition Medical Economics Cc, Montvale, N.J., 2001.
Rahman S M, Wang Y, Yotsumoto H, Cha J, Han S, Inoue S, Yanagita T, Effects of conjugated linoleic acid on serum leptin concentration, body-fat accumulation, and beta-oxidation of fatty acid in OLETF rats.
   Nutrition 2001; 17:385-90
Rebouche C J, Engel A G. Kinetic compartmental analysis of carnitine metabolism in the human carnitine deficiency syndromes. J Clin Invest 1984:73; 857-67.
Rebouche C J, Seim H. Carnitine metabolism and its regulation in microorganisms and mammals. Annu Rev Nutr 1998; 18:39-61.
Ratter A S. Carnitine and Its role in cardiovascular disease. Heart 1999; 1:108-113.
Schonekess B O, Allard M F, Lopaschuk G D. Propionyl L-carnitine improvement of hypertrophied heart function is accompanied by an increase in carbohydrate oxidation, Circ Res 1995; 77:726-34.
Sebedio J L, Gneedig S, Chardigny J M. Recent Advances in conjugated linoleic acid research. Current Opinion in Clinical Nutrition and Metabolic Care 1000; 2:499-506.
Rahman S M, Wang Y, Yotsumoto H, Cho J, Han S, Inoue S, Yanagita T. Effects of conjugated linoleic acid on serum leptin concentration, body-fat accumulation, and beta-oxidation of fatty acid in OLETF rats. Nutrition 2001; 17:385-90.

It is to be understood by those skilled in the art that the foregoing description and examples are illustrative of practicing the present invention, but are in no way limiting. Variations of the detail presented herein may be made without departing from the spirit and scope of the present invention as defined by the following claims.

1. A method of treating obesity in a patient or subject comprising coadministering to said patient or subject a synergistic effective amount of a combination of a compound according to the chemical structure: 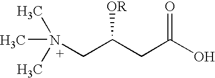
where R is H or a C₂ to C₂₀ straight or branch-chained alkanoyl group, or a pharmaceutically acceptable salt or derivative thereof in combination with conjugated linoleic acid.

2. The method according to claim 1 wherein R is a C₂ to C₈ straight or branch-chained alkanoyl group.

3. The method according to claim 1 where R is a C₂ to C₅ straight or branch-chained alkanoyl group.

4. The method according to claim 1 wherein R is an acetyl, propionyl, butyryl, valeryl or isovaleryl group.

5. The method according to claim 1 wherein said CLA comprisies an octadecadienoic acid isomer selected from the group consisting of cis-9, trans-11; cis-9, cis-11; trans-9, cis-11; trans-9, trans-11; cis-10, cis-10, trans-12; trans-10, cis-12; trans-10, trans-12 octadecadienoic acid and mixtures thereof.

6. The method according to claim 1 wherein said CLA an octadecadienoic acid isomeric selected from the group consisting of cis-11, trans-13; trans-11, cis-13; trans-11, trans-13; and cis-11, cis-13 octadecadienoic acid and mixtures thereof.

7. A method of treating a secondary condition of obesity in an obese patient wherein said secondary condition is selected from the group consisting of diabetes mellitus type I and II, high glucose levels, hyperlipidemia, hypercholesterolemia, metabolic syndrome X, kidney failure and high blood pressure in a patient or subject comprising coadministering to said patient or subject a synergistic effective amount of a combination of a compound according to the chemical structure: 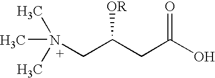
where R is H or a C₂ to C₂₀ straight or branch-chained alkanoyl group, or a pharmaceutically acceptable salt thereof in combination with conjugated linoleic acid.

8. The method according to claim 7 wherein R is a C₂ to C₈ straight or branch-chained alkanoyl group.

9. The method according to claim 7 where R is a C₂ to C₅ straight or branch-chained alkanoyl group.

10. The method according to claim 7 wherein R is an acetyl, propionyl, butyryl, valeryl or isovaleryl group.

11. The method according to claim 7 wherein said CLA comprisies an octadecadienoic acid isomer selected from the group consisting of cis-9, trans-11; cis-9, cis-11; trans-9, cis-1; trans-9, trans-11; cis-10, cis-10, trans-12; trans-10, cis-12; trans-10, trans-12 octadecadienoic acid and mixtures thereof.

12. The method according to claim 7 wherein said CLA an octadecadienoic acid isomeric selected from the group consisting of cis-11, trans-13; trans-11, cis-13; trans-11, trans-13; and cis-11, cis-13 octadecadienoic acid and mixtures thereof.

13. The method according to claim 7 wherein said condition is metabolic syndrome X.

14. The method according to claim 7 wherein said conditon is type II diabetes mellitus.

15. The method according to claim 7 wherein said condition is high blood pressure or kidney failure.

16. The method according to claim 7 wherein said synergistic amount comprises at least about 500 mg per day of carnitine and at least about 3 grams per day of conjugated linoleic acid.

17. A composition comprising a mixture of at least 100 mg of a compound according to the chemical structure: 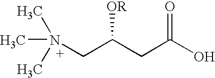
where R is H or a C₂ to C₂₀ straight or branch-chained alkanoyl group, or a pharmaceutically acceptable salt thereof in combination with at least 250 mg of conjugated linoleic acid, optionally in combination with a pharmaceutically acceptable carrier, additive or excipient.

18. The composition according to claim 17 wherein said compound comprises at least about 250 mg by weight of said composition and conjugated linoleic acid comprises at least about 500 mg by weight of said composition.

19. The composition according to claim 17 adapted for oral administration to a patient or subject.

20. The composition according to claim 17 encapsulated in a softgel capsule.

21. A method of treating obesity in a patient in need thereof, comprising administering to said patient a synergistic effective amount of a combination of carnitine or a pharmaceutically acceptable salt or derivative thereof and conjugated linoleic acid wherein said method results in said patient losing at least about 1% body weight within a period of less than about six months.

22. The method according to claim 21 wherein said subject loses at least about 2-3% body weight within a period of no more than about 2 months.

23. A method of treating obesity in a patient in need thereof, comprising administering to said patient a synergistic effective amount of a combination of carnitine or a pharmaceutically acceptable salt or derivative thereof and conjugated linoleic acid wherein said method results in the body mass index of said patient decreasing by at least about 0.5% within a period of less than about six months.

24. The method according to claim 23 wherein said body mass index of said patient decreases at least about 2-3% within a period of no more than about 2 months.
